SEQUENCE LISTING (8 sequences)

SEQ ID NO: 1 - DNA, 240 bp, Homo sapiens
ccccccccacctcctcccttgcctggagaagcaggaatgccacctcctcctccccctcttcctggtggtcctggaatccctccacctcctccatttcccggaggccctggcattcctccacctccacccggaatgggtatgcctccacctcccccatttggatttggagttcctgcagccccagttctgccatttggattaacccccaaaaagctttataagccagaggtgcagctccgg

SEQ ID NO: 2 - DNA, 240 bp, Artificial Sequence; identical to SEQ ID NO: 1
  Diaphanous cDNA

SEQ ID NO: 3 - DNA, 240 bp, Artificial Sequence; identical to SEQ ID NO: 1
  Yeast clone

SEQ ID NO: 4 - DNA, 1391 bp, Homo sapiens
ggggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgcagggcaatgaacaggaatggaaaggagaccaagtccaactaccgagtccgtgtctaccagattcctgggaagccagaaattgtagattctgcctctgaactcacggctggtgttcccaataaggtggggacatgtgtgtcagagggaagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcctggtggaaccgtaaccctgacctgtgaagtccctgcccagccctctcctcaaatccactggatgaaggatggtgtgcccttgccccttccccccagccctgtgctgatcctccctgagatagggcctcaggaccagggaacctacagctgtgtggccacccattccagccacgggccccaggaaagccgtgctgtcagcatcagcatcatcgaaccaggcgaggaggggccaactgcaggctctgtgggaggatcagggctgggaactctagccctggccctggggatcctgggaggcctggggacagccgccctgctcattggggtcatcttgtggcaaaggcggcaacgccgaggagaggagaggaaggccccagaaaaccaggaggaagaggaggagcgtgcagaactgaatcagtcggaggaacctgaggcaggcgagagtagtactggagggccttgaggggcccacagacagatcccatccatcagctcccttttctttttcccttgaactgttctggcctcagaccaactctctcctgtataatctctctcctgtataaccccaccttgccaagctttcttctacaaccagagccccccacaatgatgattaaacacctgacacatcttgca

SEQ ID NO: 5 - protein, 404 aa, Homo sapiens
GAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCRAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLALALGILGGLGTAALLIGVILWQRRQRRGEERKAPENQEEEEERAELNQSEEPEAGESSTGGP

SEQ ID NO: 6 - DNA, 5635 bp, Homo sapiens
atggagccgcccggcgggagcctggggcccggccgcgagacccgggacaagaagaagggccggagcccagatgagctgccctcggcgggcggcgacggcggcaaatctaagaaatttctggagagatttaccagcatgagaattaagaaggagaaggaaaagcccaattctgctcatagaaattcttctgcatcatatggggatgatcccacagcacagtcattgcaagatgtttcagatgaacaagtgctggttctctttgaacagatgctgctggatatgaacctgaatgaggagaaacagcaacctttgagggagaaggacatcatcatcaagagggagatggtgtcccaatacttgtacacctccaaggctggcatgagccagaaggagagctctaagtctgccatgatgtatattcaggagttgaggtcaggcttgcgggatatgcctctgctcagctgcctggagtcccttcgtgtgtctctcaacaacaaccctgtcagttgggtgcaaacatttggtgctgaaggcttggcctccttattggacattcttaaacgacttcatgatgagaaagaagagactgctgggagttacgatagccggaacaagcatgagatcattcgctgcttgaaagcttttatgaacaacaagtttggaatcaagaccatgttggagacagaagaaggaatcctactgctggtcagagccatggatcctgctgttcccaacatgatgattgatgcagctaagctgctttctgctctttgtattctaccgcagccagaggacatgaatgaaagggttttggaggcaatgacagaaagagctgagatggatgaagtggaacgtttccagccgctgctggatggattaaaaagtggaaccactattgcactgaaggttggatgcctacagctgatcaatgctctcatcacaccagcggaggaacttgacttccgagttcacatcagaagtgaactgatgcgtttggggctacatcaggtgttgcaggaccttcgagagattgaaaatgaagatatgagagtgcaactaaatgtgtttgatgaacaaggggaagaggattcctatgacctgaagggacggctggatgacattcgcatggagatggatgactttaatgaagtctttcagattctcttaaacacagtgaaggattcaaaggcagagccacacttcctttccatcctgcagcacttactcttggtccgaaatgactatgaggccagacctcagtactataagttgattgaagaatgtatttcccagatagttctgcacaagaacggggctgatcctgacttcaagtgccggcacctccagattgagattgagggattaattgatcaaatgattgataagacaaaggtggagaaatctgaagccaaagctgcagagctggaaaagaagttggactcagagttaacagcccgacatgagctacaggtggaaatgaaaaagatggaaagtgactttgagcagaagcttcaagatcttcagggagaaaaagatgcactgcattctgaaaagcagcaaattgccacagagaaacaggacctggaagcagaggtgtcccagctcacaggagaggttgccaagctgacaaaggaactggaagatgccaagaaagaaatggcttccctctctgcggcagctattactgtacctccttctgttcctagtcgtgctcctgttccccctgcccctcctttacctggtgactctggcactattattccaccaccacctgctcctggggatagtaccactcctcctcctcctccaccaccaccacctccaccacctcctttacctggaggtactgctatctctccaccccctcctttgtctggggatgctaccatccctccaccccctcctttgcctgagggtgttggcatcccttcaccctcttctttgcctggaggtactgccatccccccacctcctcctttgcctgggagtgctagaatccccccaccaccacctcctttgcctgggagtgctggaattccccccccacctcctcccttgcctggagaagcaggaatgccacctcctcctccccctcttcctggtggtcctggaatccctccacctcctccatttcccggaggccctggcattcctccacctccacccggaatgggtatgcctccacctcccccatttggatttggagttcctgcagccccagttctgccatttggattaacccccaaaaagctttataagccagaggtgcagctccggaggccaaactggtccaagcttgtggctgaggacctctcccaggactgcttctggacaaaggtgaaggaggaccgctttgagaacaatgaacttttcgccaaacttacccttaccttctctgcccagaccaagaccaagaaggatcaagaaggtggagaagaaaagaaatctgtgcaaaagaaaaaagtaaaagagttaaaggtgttggattcaaagacagcccagaatctctcaatctttttgggttccttccgcatgccctatcaagagattaagaatgtcatcctggaggtgaatgaggctgttctgactgagtctatgatccagaacctcattaagcaaatgccagagccagagcagttaaaaatgctttctgaactgaaggatgaatatgatgacctggctgagtcagagcagtttggcgtggtgatgggcactgtgccccgactgcggcctcgcctcaatgccattctcttcaagctacaattcagcgagcaagtggagaatatcaagccagagattgtgtctgtcactgctgcatgtgaggagttacgtaagagtgagagcttttccaatctcctagagattaccttgcttgttggaaattacatgaatgctggctccagaaatgctggtgcttttggcttcaatatcagcttcctctgtaagcttcgagacaccaagtccacagatcagaagatgacgttgttacacttcttggctgagttgtgtgagaatgactatcccgatgtcctcaagtttccagacgagcttgcccatgtggagaaagccagccgagtttctgctgaaaacttgcaaaagaacctagatcagatgaagaaacaaatttctgatgtggaacgtgatgttcagaatttcccagctgccacagatgaaaaagacaagtttgttgaaaaaatgaccagctttgtgaaggatgcacaggaacagtataacaagctgcggatgatgcattctaacatggagaccctctataaggagctgggcgagtacttcctctttgaccccaagaagttgtctgttgaagaatttttcatggatcttcacaattttcggaatatgtttttgcaagcagtcaaggagaaccagaagcggcggaagacagaagaaaagatgaggcgagcaaaactagccaaggagaaggcagagaaggagcggctagagaagcagcagaagagagagcaactcatagacatgaatgcagagggcgatgagacaggtgtgatggacagtcttctagaagccctgcagtcaggggcagcattccgacggaagagagggccccgtcaagccaacaggaaggccgggtgtgcagtcacatctctgctagcttcggagctgaccaaggatgatgccatggctgctgttcctgccaaggtgtccaagaacagtgagacattccccacaatccttgaggaagccaaggagttggttggccgtgcaagctaatgtgggtcctgtgaccgcggcagctcctcagcggagccgcagactgtcctgccctgcagcatgtgcctaaaggctcaaggggatattcctctggggtggccactcccaccaccctgaccctgtctttctctctggcctgctgctctctcaacatcacatacagcttcagctgcctggaggccagaaggaaagggcagtgcaggggaggcctgagcccgacttagccagccctggctgttgtattaccaaagcagggtccatgtttgctgccttaaccctgtctcctctctgttactcagagggcctcatctcagacaaggcccagcctgctttttctcagccctgactttctaatgggctttcccccctaggtcagtcttgctggatttgtgcttttcttttgtggtttctctggccctgagaatagcatggggcttgtaaacctttgggctagatccctcctttcattgctgttgtctctgctcttccctctcctggctgtggttatttattattagtggtgtggcactgggagctgctcctaaggaagcagggagcaaatcccacctttaccccaccttcctgggaaaggcctccaaagcaaaggatctggaccagtttccctgctgtgctgtggcccaggccagagcctgtgggcaggcaggcagggcatagcgacagtgtgggacctgcccccagcttctgccacgctttatgcccttgcctctctggacgctctgcaccaaccccaggctactgagccaccttccctcctcatgccttccctgagctttggtgcatctcatctggactatgggttgtactgtgaccatcccaacacctcaccctctgtctacaaggaaatgggaggtggagggttgtactgtgaccatcccaacacctcaccctctgtctacaaggaaatgggaggtggagcctcctggctgagaaattgttttgcaaatggatctatttttgtatgaaaaaaaaaatttttttaaagaaaactgttccttccccctttcccctccataatgtaagaagctttggtggcaggttacagagttctgggatttcttctcacaggcccaatcctgaatgtgcccctggaccttctggacccttgagtccaaggcagatcctctctcccagggaatccgacacaggaggaaccccttctctggttgagctgggccaggcctaagagtagcaggaactctaagaccacagagttttttataaatgtataaatgtatcaagccaaatgtgcagatgctaactggacattctggggaactgggcaccaggagtgccttcatacactgtaccccagctctcttctaaaagagaagtgggtgggcacactgaactgtttggtggccccaaccacaggaagctgcaattctgtggcttagggtgatacttttgccctccttgtgcccctctcagctttccatccccagctaggaagaaagaatggcactcttggcttggcccagaattagagttattagagcaagagagagcttaggaagcatgagggcaactatagtgaggccttattgccaggagggagggttttggttgctggcgcttgtgtataaaggggcaagagcagctcctttggactattcctgggaggactctgatgcagggcgtctgttgctcccctgggtcacctcctccctgctcgctgacatctggggctttgaccctttcttttttaatctacttttgctaagatgcatttaataaaaaaaaagagagagagagagaggtgtgagggacaaaatgcaaacctatttcccttgcctcataggcttctgggatgtcatcacctccagtttgttggttttgtttccaactgttaataaagcattgaaacagt

SEQ ID NO: 7 - protein, 1248 aa, Homo sapiens
MEPPGGSLGPGRGTRDKKKGRSPDELPSAGGDGGKSKKFLERFTSMRIKKEKEKPNSAHRNSSASYGDDPTAQSLQDVSDEQVLVLFEQMLLDMNLNEEKQQPLREKDIIIKREMVSQYLYTSKAGMSQKESSKSAMMYIQELRSGLRDMPLLSCLESLRVSLNNNPVSWVQTFGAEGLASLLDILKRLHDEKEETAGSYDSRNKHEIIRCLKAFMNNKFGIKTMLETEEGILLLVRAMDPAVPNMMIDAAKLLSALCILPQPEDMNERVLEAMTERAEMDEVERFQPLLDGLKSGTTIALKVGCLQLINALITPAEELDFRVHIRSELMRLGLHQVLQDLREIENEDMRVQLNVFDEQGEEDSYDLKGRLDDIRMEMDDFNEVFQILLNTVKDSKAEPHFLSILQHLLLVRNDYEARPQYYKLIEECISQIVLHKNGADPDFKCRHLQIEIEGLIDQMIDKTKVEKSEAKAAELEKKLDSELTARHELQVEMKKMESDFEQKLQDLQGEKDALHSEKQQIATEKQDLEAEVSQLTGEVAKLTKELEDAKKEMASLSAAAITVPPSVPSRAPVPPAPPLPGDSGTIIPPPPAPGDSTTPPPPPPPPPPPPPLPGGTAISPPPPLSGDATIPPPPPLPEGVGIPSPSSLPGGTAIPPPPPLPGSARIPPPPPPLPGSAGIPPPPPPLPGEAGMPPPPPPLPGGPGIPPPPPFPGGPGIPPPPPGMGMPPPPPFGFGVPAAPVLPFGLTPKKLYKPEVQLRRPNWSKLVAEDLSQDCFWTKVKEDRFENNELFAKLTLTFSAQTKTKKDQEGGEEKKSVQKKKVKELKVLDSKTAQNLSIFLGSFRMPYQEIKNVILEVNEAVLTESMIQNLIKQMPEPEQLKMLSELKDEYDDLAESEQFGVVMGTVPRLRPRLNAILFKLQFSEQVENIKPEIVSVTAACEELRKSESFSNLLEITLLVGNYMNAGSRNAGAFGFNISFLCKLRDTKSTDQKMTLLHFLAELCENDYPDVLKFPDELAHVEKASRVSAENLQKNLDQMKKQISDVERDVQNFPAATDEKDKFVEKMTSFVKDAQEQYNKLRMMHSNMETLYKELGEYFLFDPKKLSVEEFFMDLHNFRNMFLQAVKENQKRRETEEKMRRAKLAKEKAEKERLEKQQKREQLIDMNAEGDETGVMDSLLEALQSGAAFRRKRGPRQANRKAGCAVTSLLASELTKDDAMAAVPAKVSKNSETFPTILEEAKELVGRAS

SEQ ID NO: 8 - protein, 41 aa, Homo sapiens
QRRQRRGEERKAPENQEEEEERAELNQSEEPEAGESSTGGP

(19) United States
(12) Patent Application Publication
(10) Pub. No.: US 2009/0220484 A1
(43) Pub. Date: Sep. 3, 2009

(54) Rage/Diaphanous Interaction and Related Compositions and Methods

(76) Inventors: Ann Marie Schmidt, Franklin Lakes, NJ (US); Barry Hudson, New York, NY (US)

Correspondence Address: COOPER & DUNHAM, LLP, 30 Rockefeller Plaza, 20th Floor, NEW YORK, NY, 10112, US

(21) Appl. No.: 11/886,651

(22) PCT Filed: Mar. 17, 2006
(86) PCT No.: PCT/US2006/010045

§ 371 Date: Feb. 27, 2009

Related U.S. Application Data
(60) Provisional application No. 60/662,618, filed on Mar. 17, 2005.

(51) Int. Cl. A61K 39/395; C07K 16/18; C12N 15/11; C12N 15/00; C12N 5/06; C12N 1/21; C12N 1/19

(52) U.S. Cl. 424/130.1; 530/387.1; 536/23.53; 435/320.1; 435/326; 435/252.3; 435/252.4; 435/254.11

(57) ABSTRACT

This invention provides a polypeptide consisting essentially of all or a portion of the cytoplasmic domain of RAGE. This invention further provides a polypeptide consisting essentially of a portion of Diaphanous that binds to the cytoplasmic domain of RAGE. Additionally, this invention provides related nucleic acids, vectors, cells and methods.

This application claims the benefit of U.S. Provisional Application No. 60/662,618, filed Mar. 17, 2005, the contents of which are incorporated hereby by reference into the subject application.

This invention was made with support under United States Government Grant Nos. CA87677 and HL60901 from the National Institutes of Health. Accordingly, the United States Government has certain rights in the subject invention.

Throughout the application, various publications are referenced. Full citations for these publications may be found immediately preceding the claims. The disclosures of these publications are hereby incorporated by reference into this application in order to more fully describe the state of the art as of the date of the invention described and claimed herein.

### BACKGROUND OF THE INVENTION

Mammalian Diaphanous proteins are orthologues of the product of the gene Diaphanous in Drosophila first described for its critical role in mediating cytokinesis in the fly. Lynch and colleagues identified the mammalian orthologue and showed that a mutation in the gene encoding human Diaphanous caused nonsyndromic deafness. To date, this is the only human “disease” setting in which the molecule has been implicated.

The biology of Diaphanous is based on the domains that make up the protein Diaphanous. First, there is an autoactivation domain; this is followed by a Rho binding domain, followed by an FH1, and, lastly an FH2 domain (FH=formin homology). The key biological properties of Diaphanous based on the functions of these domains are described below.

First, Diaphanous is a ligand for profilin and target of Rho GTPases—key roles for these pathways are implicated in polymerization of the activation cytoskeleton. These considerations indicate that an essential function of this molecule is to bridge signaling pathways (Rho GTPases) that are involved in cellular motility and migration.

Second, recent studies suggest that in addition to these roles in the actin cytoskeleton, a specific function of Diaphanous is regulation of microtubules. Microtubules play central roles in fundamental aspects of cellular stabilization and further, interaction with the actin cytoskeleton. Microtubules may be involved in key biological functions of cell-cell contact (such as with inflammatory cells in the adaptive immune response).

Third, Diaphanous contains Rho binding domains. One of these Rho GTPases is rac1. Rac 1 is involved not only in interaction with the actin cytoskeleton, but, also, it is a key component of the enzyme NADPH oxidase. This enzyme contains multiple components that must be fully assembled at the cell surface in order for it to be operative. NADPH oxidase functions by generating reactive oxygen species.

### SUMMARY OF THE INVENTION

This invention provides a polypeptide consisting essentially of all or a portion of the cytoplasmic domain of RAGE.

This invention also provides a pharmaceutical composition comprising (a) all or a portion of the cytoplasmic domain of RAGE and (b) a pharmaceutically acceptable carrier.

This invention further provides a polypeptide consisting essentially of a portion of Diaphanous that binds to the cytoplasmic domain of RAGE.

This invention further provides a pharmaceutical composition comprising (a) a portion of Diaphanous that binds to the cytoplasmic domain of RAGE and (b) a pharmaceutically acceptable carrier.

This invention further provides a nucleic acid that encodes a polypeptide consisting essentially of all or a portion of the cytoplasmic domain of RAGE.

This invention further provides a nucleic acid encoding a polypeptide consisting essentially of a portion of Diaphanous that binds to the cytoplasmic domain of RAGE.

This invention further provides an expression vector comprising a nucleic acid that encodes a polypeptide consisting essentially of all or a portion of the cytoplasmic domain of RAGE.

This invention further provides an expression vector comprising a nucleic acid that encodes a polypeptide consisting essentially of a domain of Diaphanous that binds to the cytoplasmic domain of RAGE.

This invention further provides a method for inhibiting binding between Diaphanous and the cytoplasmic domain of RAGE comprising contacting Diaphanous and the cytoplasmic domain of RAGE with an agent that, under suitable conditions, inhibits binding therebetween.

This invention further provides method for identifying an agent that inhibits binding between Diaphanous and the cytoplasmic domain of RAGE comprising (a) contacting Diaphanous and the cytoplasmic domain of RAGE with the agent under conditions that would permit binding between Diaphanous and the cytoplasmic domain of RAGE in the absence of the agent, (b) after a suitable period of time, determining the amount of Diaphanous bound to the cytoplasmic domain of RAGE and (c) comparing the amount of Diaphanous bound to the cytoplasmic domain of RAGE determined in step (b) with the amount of Diaphanous bound to the cytoplasmic domain of RAGE in the absence of the agent, whereby a lower amount of binding in the presence of the agent indicates that the agent inhibits the binding between Diaphanous and the cytoplasmic domain of RAGE.

Finally, this invention provides a method for treating a RAGE-related disorder in a subject afflicted therewith comprising administering to the subject a therapeutically effective amount of an agent that inhibits the binding between Diaphanous and the cytoplasmic domain of RAGE.

### BRIEF DESCRIPTION OF THE FIGURES

FIG. 1

FIG. 1 shows a schematic diagram indicating that RAGE is a multi-ligand receptor expressed by many cell types.

FIG. 2

FIG. 2 shows experimental results indicating the blockade of RAGE in apoE null diabetic mice (23).

FIG. 3

FIG. 3 shows experimental results indicating that the blockade of RAGE diminishes albuminuria in diabetic db/db mice (24).

FIG. 4

FIG. 4 shows the expression of RAGE to enhanced degrees in human carotid endarterectomy samples (25).

FIG. 5

FIG. 5 shows a schematic illustration of how RAGE signaling is suppressed when the cytoplasmic domain of RAGE is removed (i.e., the so-called DN or dominant negative RAGE).

FIG. 6

FIG. 6 shows experimental data relating to ligand-RAGE activation of MAPkinases. In contrast, there is no effect on RAGE signaling when BSA=albumin (26).

FIG. 7

FIG. 7 shows images of the actin cytoskeleton. Cells expressing full-length functional RAGE (middle panel) have organized structures in the context of the actin cytoskeleton. In contrast, cells expressing DN RAGE (no RAGE signaling) have a very disorganized cytoskeleton (right panel).

FIG. 8

FIG. 8 shows data indicating that transgenic mice expressing DN RAGE in SMC have decreased neointimal expansion upon arterial injury (27).

FIG. 9

FIG. 9 shows a schematic illustration linking RAGE signaling to inflammation, cell proliferation and cytoskeletal regulation.

FIG. 10

FIG. 10 shows the sequence results of yeast 2 hybrid experiments (SEQ ID NOs: 1-3).

FIG. 11

FIG. 11 shows a schematic illustration of Diaphanous and its domains (RBD, FH1 and FH2).

FIG. 12

His-tagged RAGE tail and Myc-tagged Diaphanous were constructed, and then transfected into cells. Simple western blots (WB) were performed using anti-his IgG (left panel) and anti-myc IgG (right panel). The panels indicate that his-RAGE tail and myc-Diaphanous are expressing in the cells. In each gel, the marker lanes are lane 1.

FIG. 13

FIG. 13 shows data indicating that the RAGE tail interacts with Diaphanous. Top: Cells were transfected with his-RAGE tail (lane 1), his-RAGE tail+myc Diaphanous (lane 2) and myc-Diaphanous (lane 3). IP was performed with anti-his IgG and western blot with anti-myc IgG. The band in lane 2 indicates that the cytosolic domain of RAGE interacts with Diaphanous. Lanes 1 and 3 are negative controls Bottom: Cells were transfected with his-RAGE tail and IP was performed with anti-his IgG. This panel indicates that the his-RAGE tail is expressing in lanes 1 and 2 (relevant to same lanes in top panel).

FIG. 14

FIG. 14 shows cells transfected with full-length human RAGE or DN RAGE. In lanes 1 and 2, IP was performed with anti-RAGE IgG and blotted with Diaphanous. A band was present in lane 1, but not in the DN RAGE lane (no tail). This indicates that Diaphanous interacts with RAGE tail, but not other regions. The right side of the panel indicates that Diaphanous is expressed well in cells transfected with either full-length RAGE or DN RAGE. DN RAGE does not change Diaphanous expression.

FIG. 15

FIG. 15 shows results from confocal microscopy further indicating that RAGE tail interacts with Diaphanous. Top 3 lanes: Cells transfected with mock vector (no RAGE) shows small amounts of RAGE expressing endogenously. In the top right panel, cells expressing Diaphanous indicate co-localization of RAGE with Diaphanous. Middle 3 lanes: Cells transfected with full-length RAGE display much stronger RAGE staining and co-localization with Diaphanous. Bottom 3 lanes: Cells transfected with DN RAGE (no tail) display much less co-localization with Diaphanous.

FIG. 16

Mutants of the RAGE tail were made and expressed in cells. Full indicates a cell expressing full-length RAGE with the normal tail region. ¾ indicates a cell expressing RAGE with only ¾ of the RAGE tail present. ½ indicates a cell expressing RAGE with only ½ of the RAGE tail present. ¼ indicates a cell expressing RAGE with only ¼ of the RAGE tail present. DN indicates a cell expressing RAGE with no RAGE tail present.

FIG. 17

FIG. 17 shows data indicating the domains of Diaphanous mutants that have been generated to date.

FIG. 18

FIG. 18 shows data indicating that RAGE ligands stimulate generation for reactive oxygen species. Much less stimulation is observed in DN RAGE cells, indicating that RAGE signaling is essential for ligand-stimulated reactive oxygen species.

FIG. 19

FIG. 19 shows the full nucleic acid sequence encoding human RAGE (Genbank No. M91211) (SEQ ID NO: 4).

FIG. 20

FIG. 20 shows the full amino acid sequence of human PAGE (Genbank No. AAA03574) (SEQ ID NO: 5).

FIGS. 21A-D

FIGS. 21A-D show the full nucleic acid sequence of human Diaphanous. (Genbank No. AF051782) (SEQ ID NO: 6).

FIG. 22

FIG. 22 shows the amino acid sequence of human Diaphanous (Genbank No. AACA05373) (SEQ ID NO: 7).

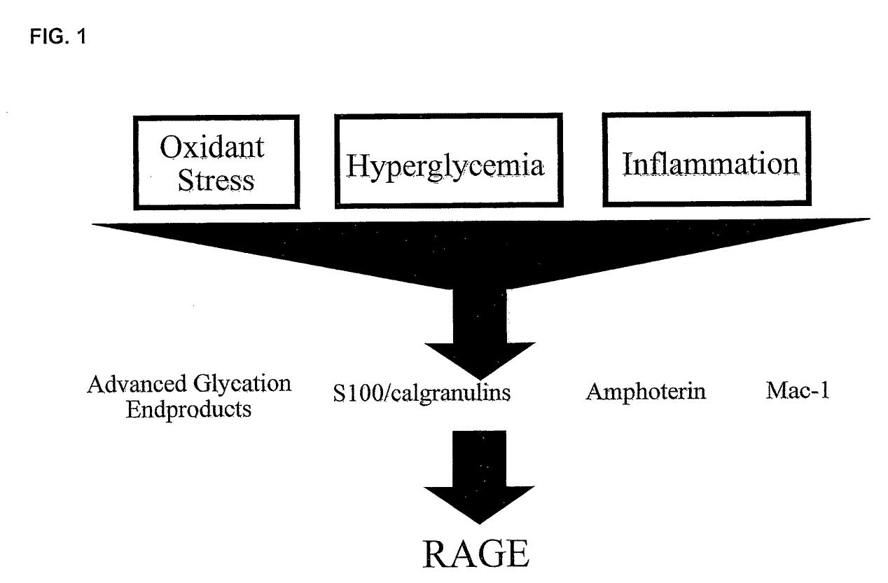

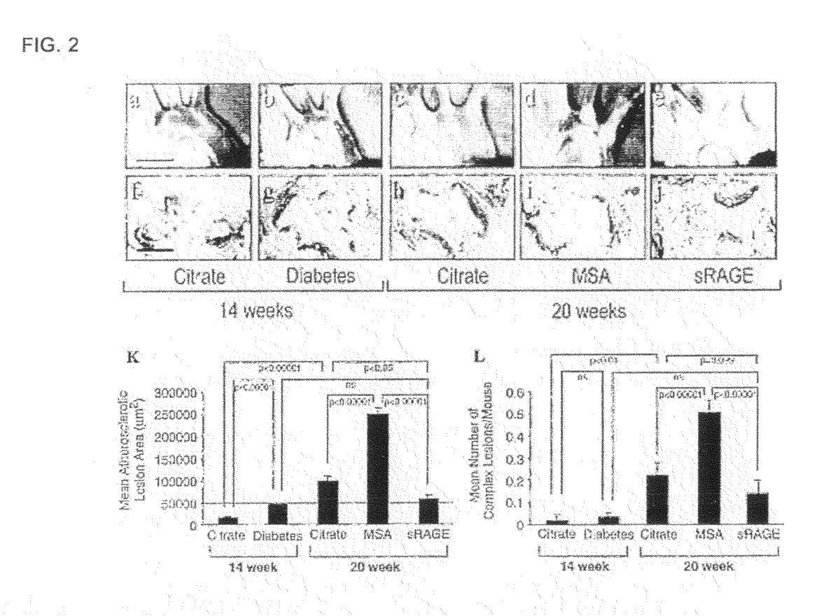

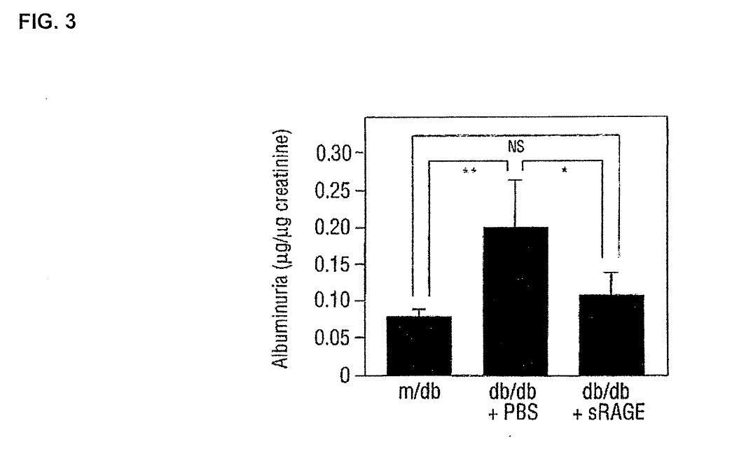

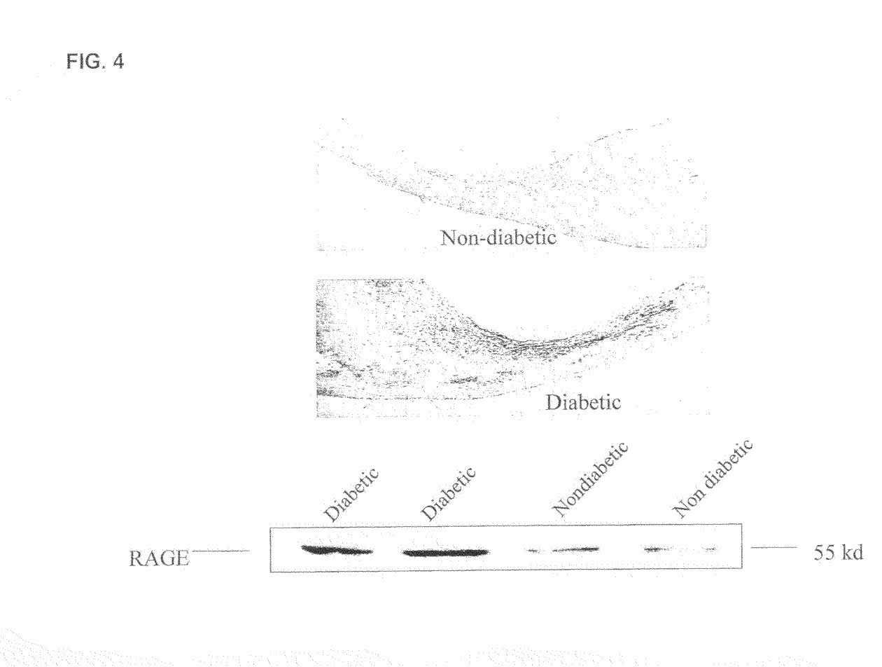

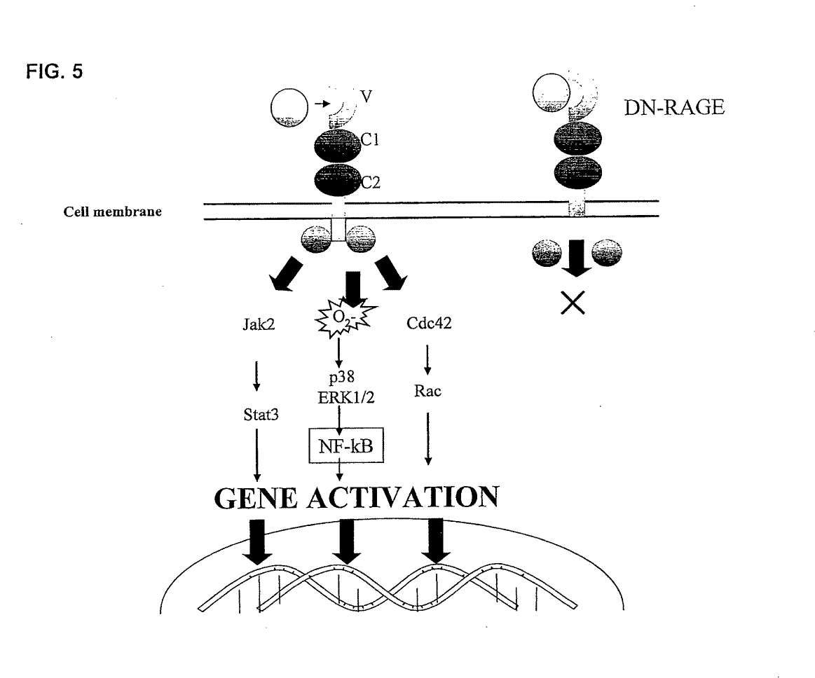

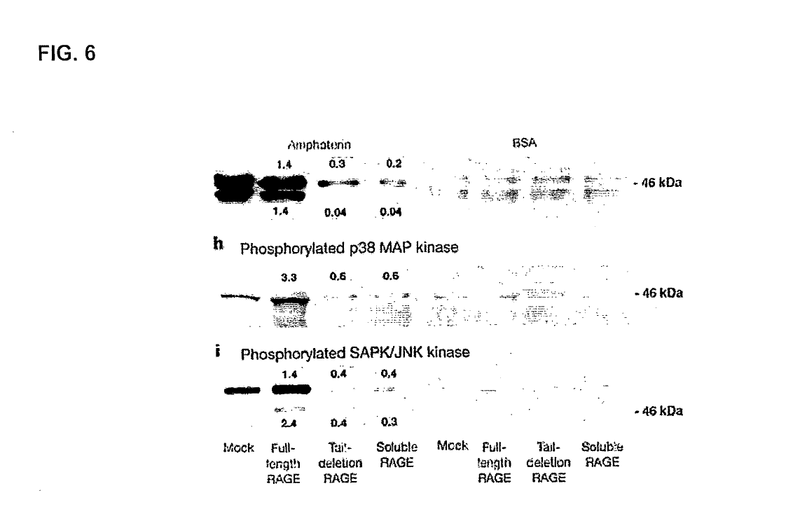

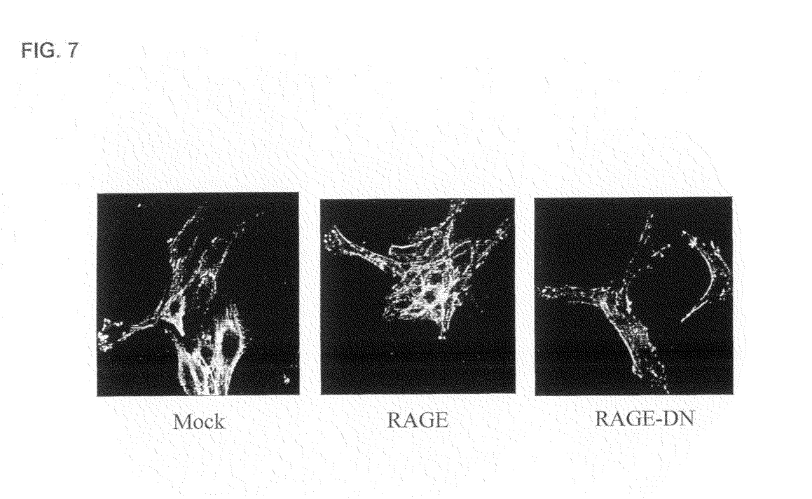

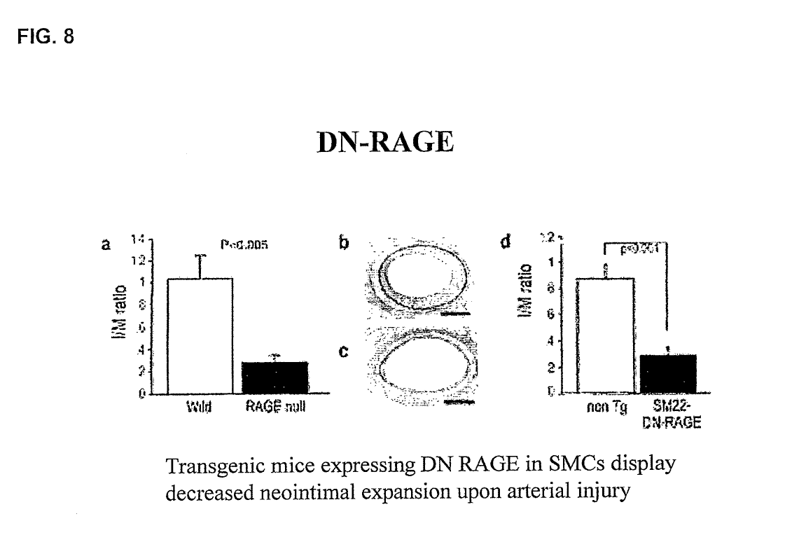

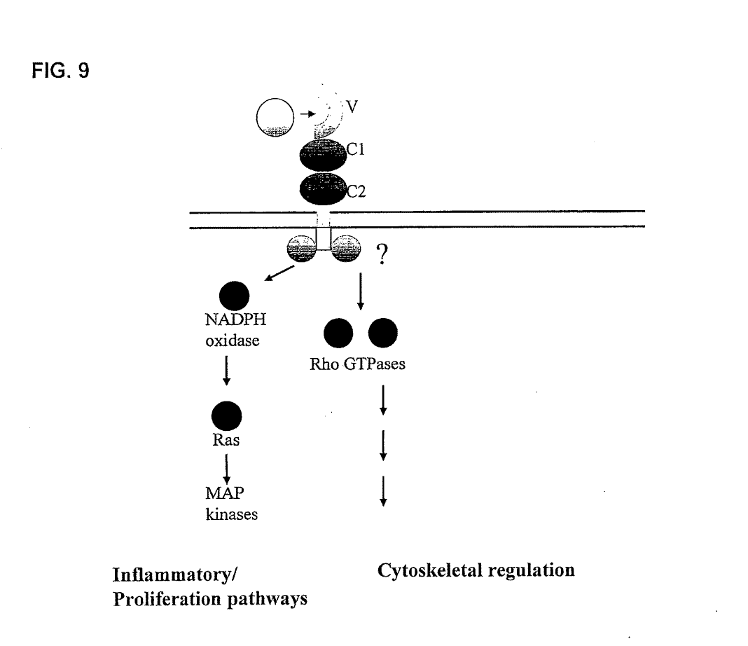

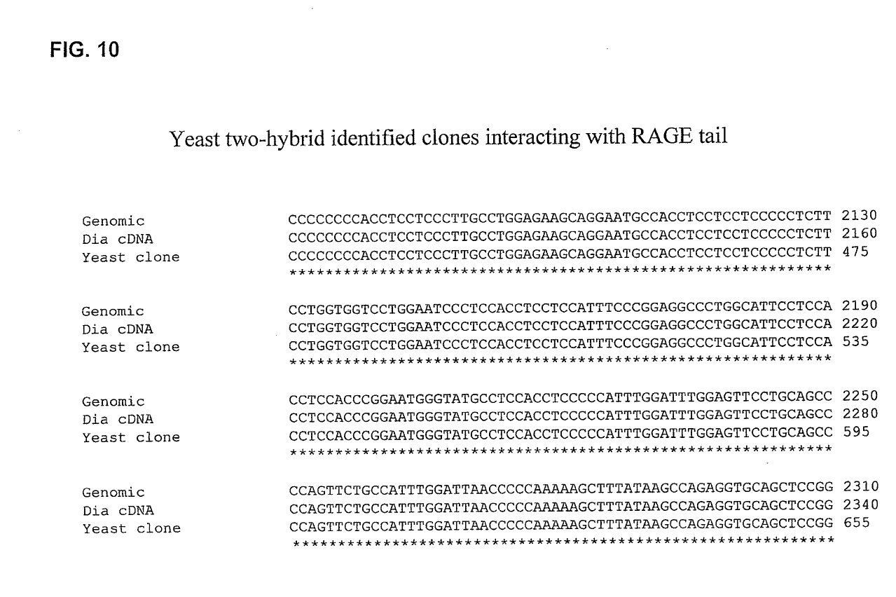

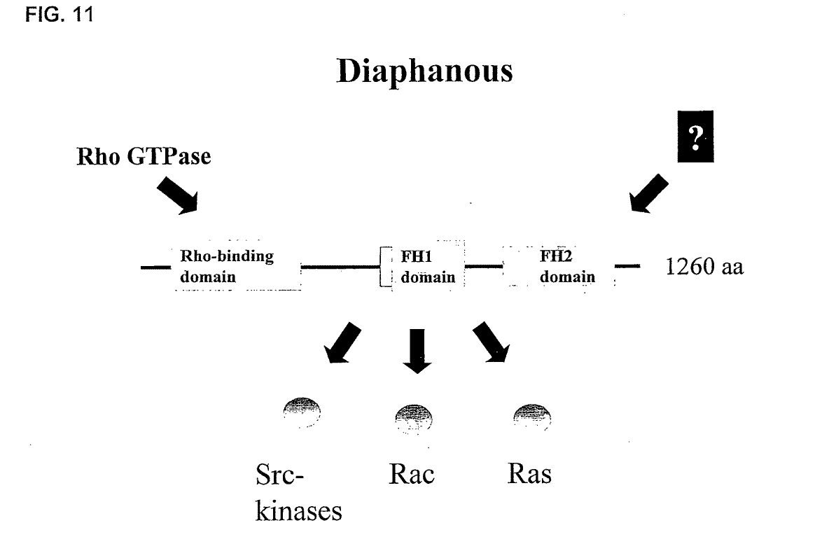

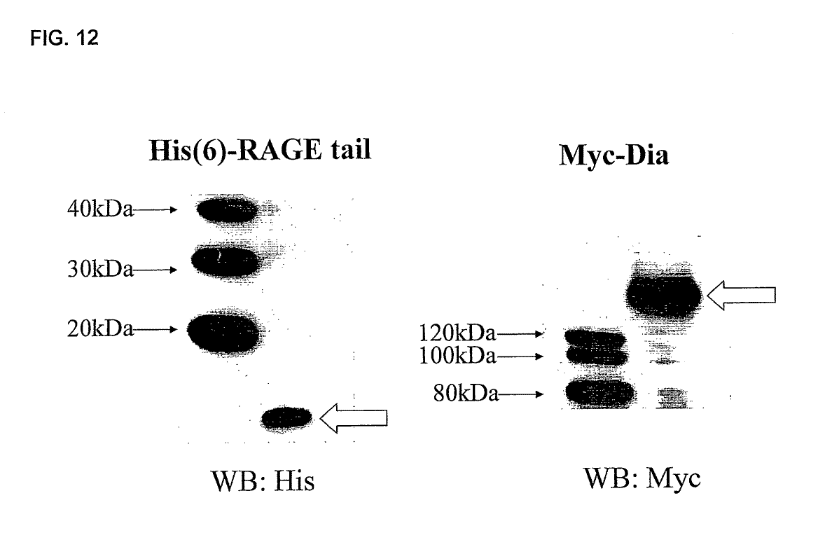

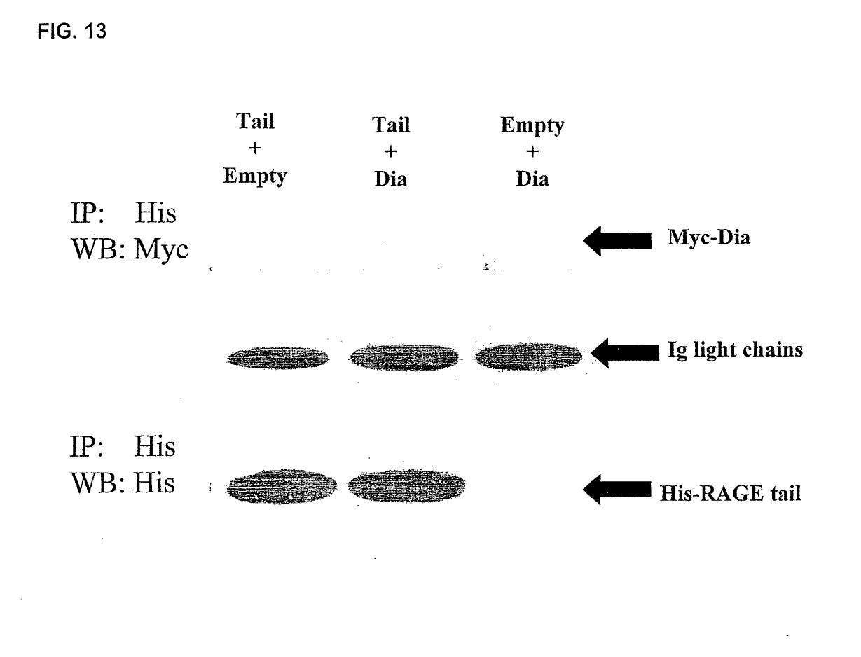

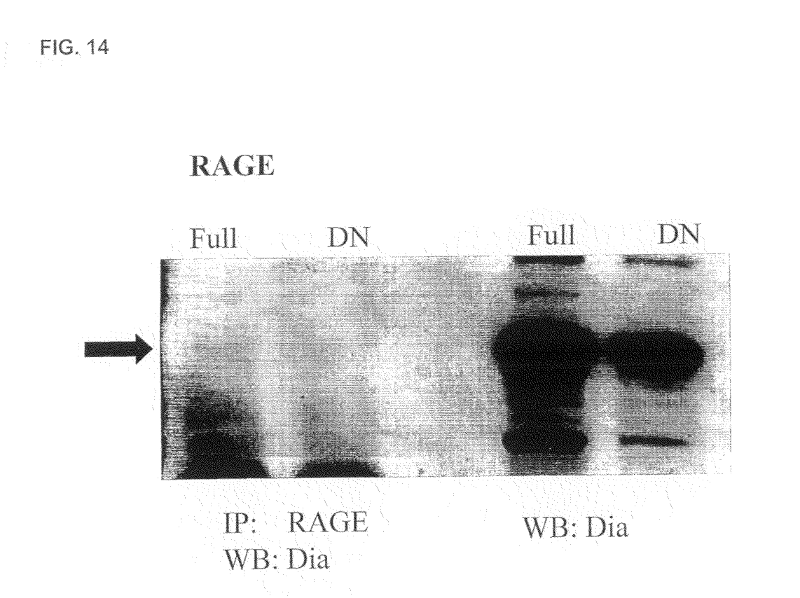

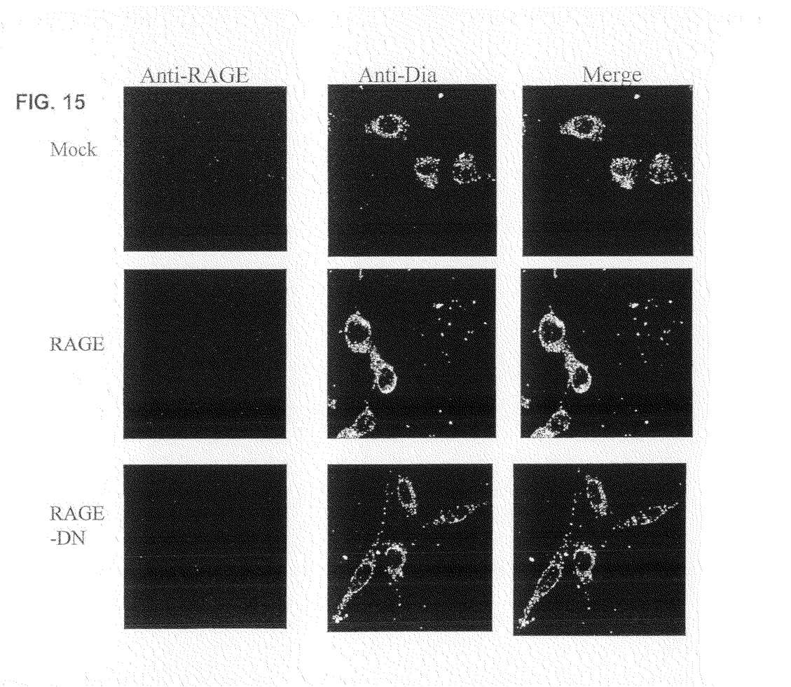

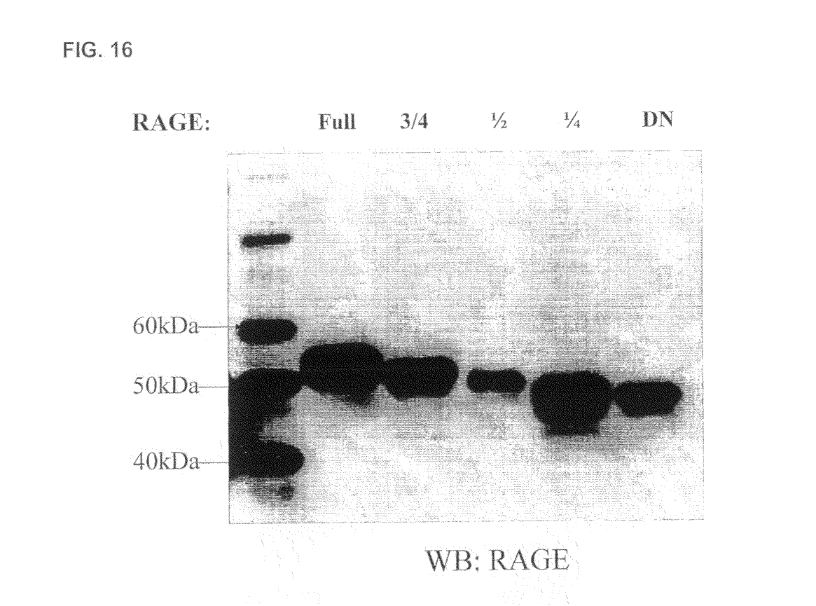

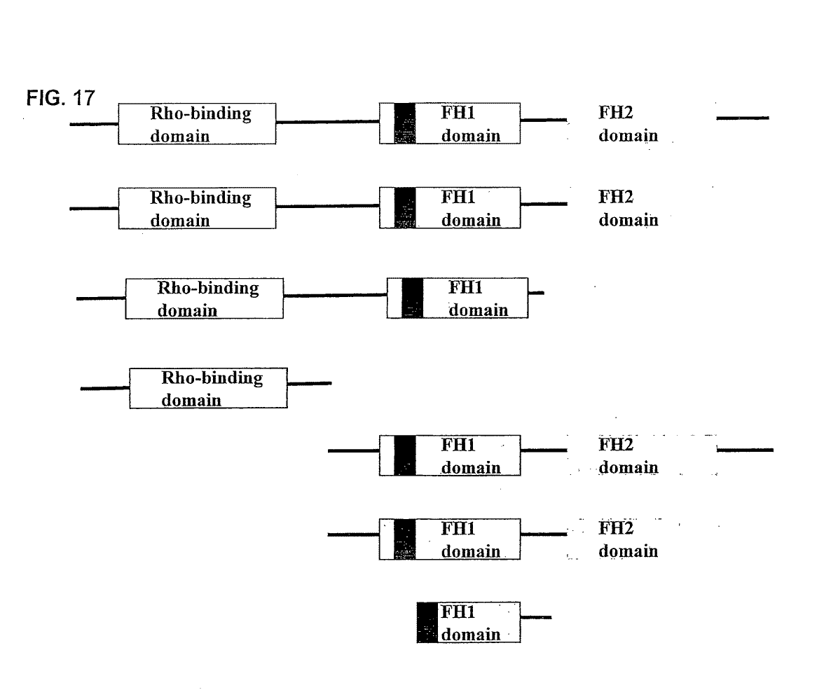

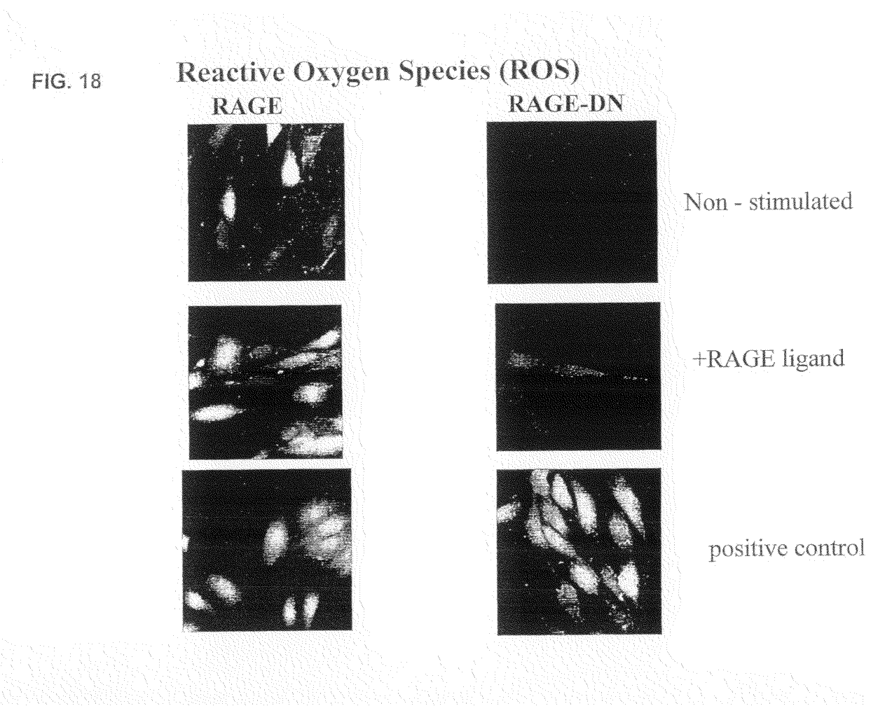

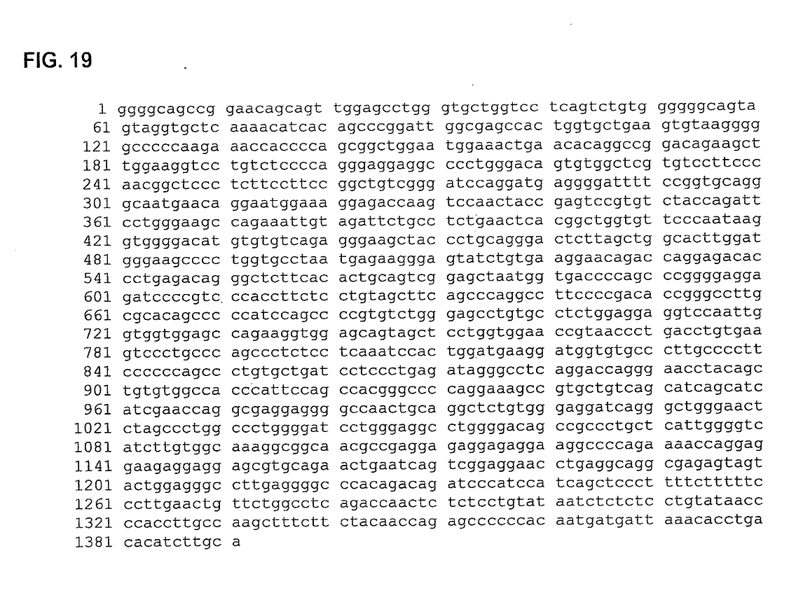

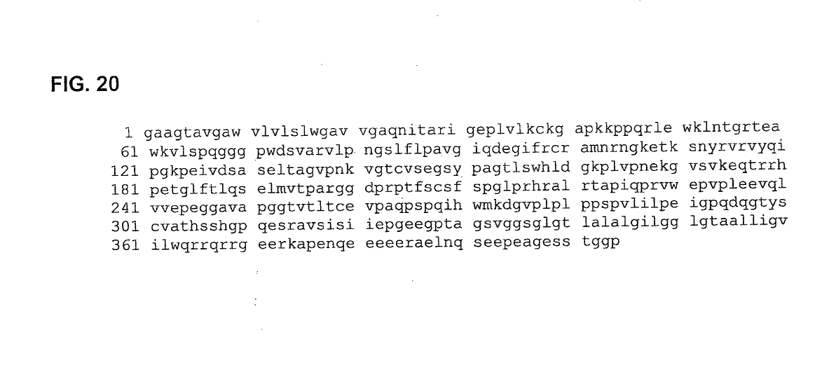

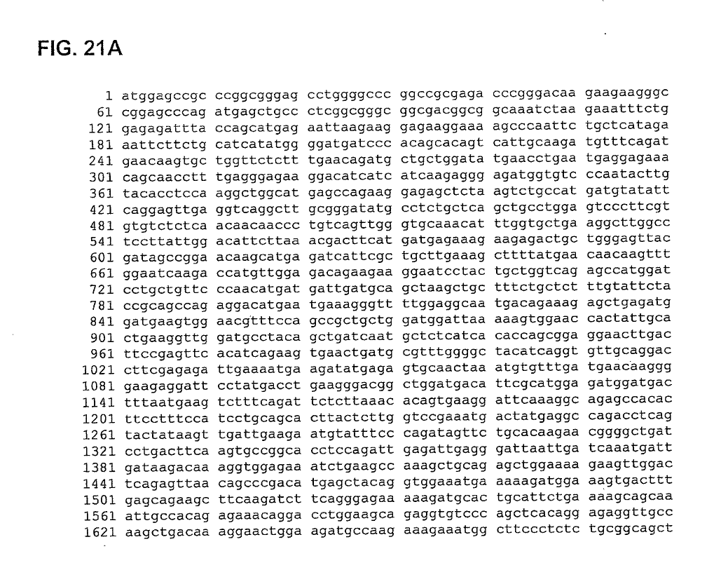

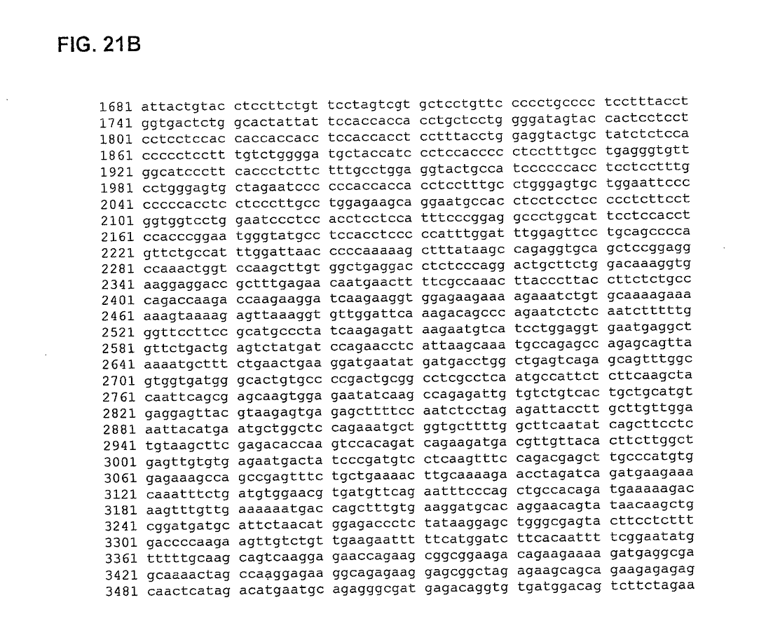

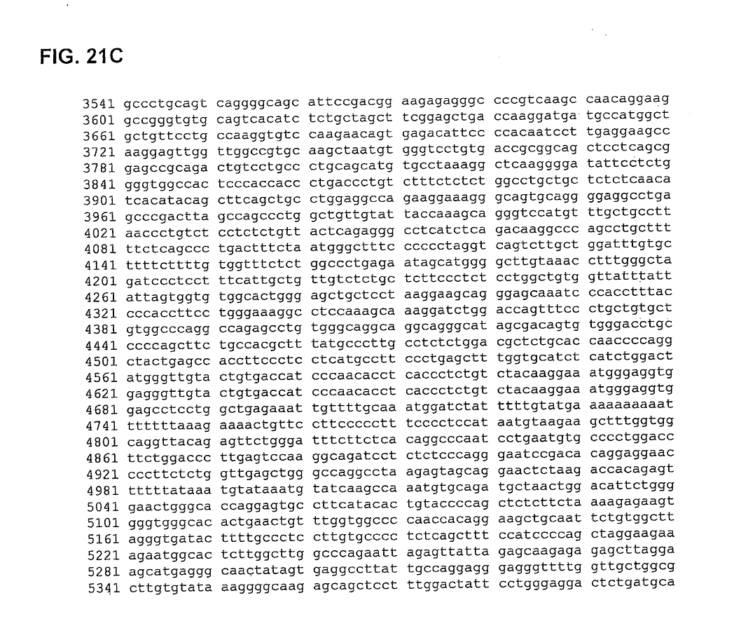

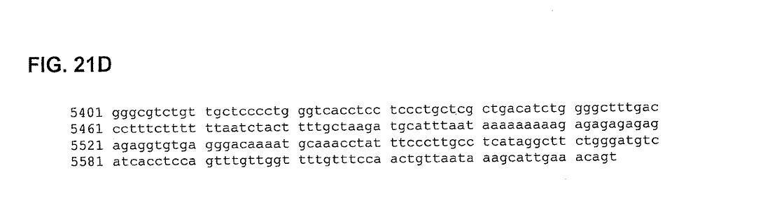

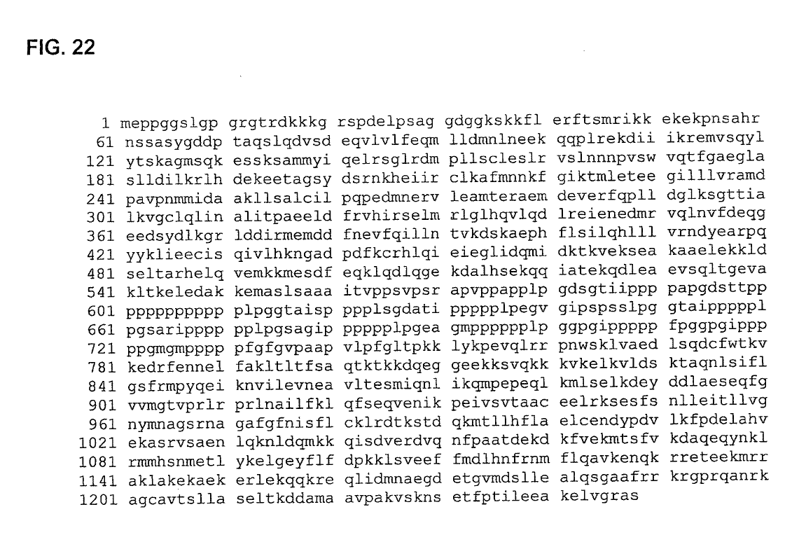

### DETAILED DESCRIPTION OF THE INVENTION

### Terms

“Administering” an agent can be effected or performed using any of the various methods and delivery systems known to those skilled in the art. The administering can be performed, for example, intravenously, orally, nasally, via the cerebrospinal fluid, via implant, transmucosally, transdermally, intramuscularly, and subcutaneously. The following delivery systems, which employ a number of routinely used pharmaceutically acceptable carriers, are only representative of the many embodiments envisioned for administering compositions according to the instant methods.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprolactones and PLGA's). Implantable systems include rods and discs, and can contain excipients such as PLGA and polycaprolactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

“Agent” shall mean any chemical entity, including, without limitation, a glycomer, a protein, an antibody, a lectin, a nucleic acid, a small molecule, and any combination thereof. Examples of possible agents include, but are not limited to, a ribozyme, a DNAzyme and an siRNA molecule.

“Antibody” shall include, by way of example, both naturally occurring and non-naturally occurring antibodies. Specifically, this term includes polyclonal and monoclonal antibodies, and antigen-binding fragments (e.g., Fab fragments) thereof. Furthermore, this term includes chimeric antibodies (e.g., humanized antibodies) and wholly synthetic antibodies, and antigen-binding fragments thereof.

“Bacterial cell” shall mean any bacterial cell. One example of a bacterial cell is E. coli.

“Consisting essentially of”, in one embodiment with respect to the cytoplasmic domain of RAGE, means not containing any of the transmembrane or extracellular domain of RAGE. In another embodiment with respect to the FH1 domain of Diaphanous, this term means not containing any other portion of Diaphanous.

“Cytosolic” and “cytoplasmic” are used synonymously with respect to RAGE, and refers to the tail portion of RAGE, i.e., the domain corresponding to amino acids residues 364-404 of the human RAGE amino acid sequence (having the sequence QRRQRRGEERKAPENQEEEEERAELNQSEEPEAGESSTGGP; SEQ ID NO: 8).

“Domain”, with respect to a region of a polypeptide, is used synonymously with “portion.”

“DNAzyme” shall mean a catalytic nucleic acid that is DNA or whose catalytic component is DNA, and which specifically recognizes and cleaves a distinct target nucleic acid sequence, which can be either DNA or RNA. Each DNAzyme has a catalytic component (also referred to as a “catalytic domain”) and a target sequence-binding component consisting of two binding domains, one on either side of the catalytic domain.

“Expression vector” shall mean a nucleic acid encoding a nucleic acid of interest and/or a protein of interest, which nucleic acid, when placed in a cell, permits the expression of the nucleic acid or protein of interest. For example, a bacterial expression vector includes a promoter such as the lac promoter and for transcription initiation the Shine-Dalgarno sequence and the start codon AUG. Similarly, a eukaryotic expression vector includes a heterologous or homologous promoter for RNA polymerase II, a downstream polyadenylation signal, the start codon AUG and a termination codon for detachment of the ribosome. Such vectors may be obtained commercially or assembled from the sequences described in methods well-known in the art.

“Inhibiting” the binding between Diaphanous and the cytoplasmic domain of RAGE shall mean either lessening the degree of such binding, or preventing the binding entirely. In one embodiment, inhibiting the binding between Diaphanous and the cytoplasmic domain of RAGE means preventing the binding entirely.

“Isolated nucleic acid”, in one embodiment, means the nucleic acid free from other nucleic acid. In another embodiment, the subject nucleic acid encoding a polypeptide consisting essentially of all or a part of the cytoplasmic domain of RAGE is isolated if it is free from any nucleic acid encoding a different polypeptide. Isolated nucleic acid can be obtained using known methods.

“Mammalian cell” shall mean any mammalian cell. Mammalian cells include, without limitation, cells which are normal, abnormal and transformed, and are exemplified by neurons, epithelial cells, muscle cells, blood cells, immune cells, stem cells, osteocytes, endothelial cells and blast cells.

“Nucleic acid” shall mean any nucleic acid molecule, including, without limitation, DNA (e.g., cDNA), RNA and hybrids thereof. The nucleic acid bases that form nucleic acid molecules can be the bases A, C, G, T and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, N.J., USA).

“Polypeptide” and “protein” are used interchangeably herein, and each means a polymer of amino acid residues. The amino acid residues can be naturally occurring or chemical analogues thereof. Polypeptides and proteins can also include modifications such as glycosylation, lipid attachment, sulfation, hydroxylation, and ADP-ribosylation.

“RAGE” shall mean receptor for advanced glycation endproducts. RAGE can be, for example, from human or any other species which produces this protein. The nucleotide and protein (amino acid) sequences for RAGE are known (Genbank Nos. M91211 and AAA03574, respectively) The following references, inter alia, also provide these sequences: Schmidt et al, J. Biol. Chem., 267:14987-97, 1992; and Neeper et al, J. Biol. Chem., 267:14998-15004, 1992. Additional RAGE sequences (DNA sequences and translations) are available from GenBank.

“RAGE-related disorder” means any disorder whose cause or symptoms are mediated, in whole or in part, by RAGE.

“Ribozyme” shall mean a catalytic nucleic acid molecule which is RNA or whose catalytic component is RNA, and which specifically recognizes and cleaves a distinct target nucleic acid sequence, which can be either DNA or RNA. Each ribozyme has a catalytic component (also referred to as a “catalytic domain”) and a target sequence-binding component consisting of two binding domains, one on either side of the catalytic domain.

“siRNA” shall mean small interfering ribonucleic acid. Methods of designing and producing siRNA to decrease the expression of a target protein are well known in the art.

“Subject” shall mean any animal, such as a human, non-human primate, mouse, rat, guinea pig or rabbit.

“Therapeutically effective amount” means an amount sufficient to treat a subject afflicted with a disorder or a complication associated with a disorder. The therapeutically effective amount will vary with the subject being treated, the condition to be treated, the agent delivered and the route of delivery. A person of ordinary skill in the art can perform routine titration experiments to determine such an amount. Depending upon the agent delivered, the therapeutically effective amount of agent can be delivered continuously, such as by continuous pump, or at periodic intervals (for example, on one or more separate occasions). Desired time intervals of multiple amounts of a particular agent can be determined without undue experimentation by one skilled in the art. In one embodiment, the therapeutically effective amount is from about 1 mg of agent/subject to about 1 g of agent/subject per dosing. In another embodiment, the therapeutically effective amount is from about 10 mg of agent/subject to 500 mg of agent/subject. In a further embodiment, the therapeutically effective amount is from about 50 mg of agent/subject to 200 mg of agent/subject. In a further embodiment, the therapeutically effective amount is about 100 mg of agent/subject. In still a further embodiment, the therapeutically effective amount is selected from 50 mg of agent/subject, 100 mg of agent/subject, 150 mg of agent/subject, 200 mg of agent/subject, 250 mg of agent/subject, 300 mg of agent/subject, 400 mg of agent/subject and 500 mg of agent/subject.

“Treating” a disorder shall mean slowing, stopping or reversing the disorder's progression. In the preferred embodiment, treating a disorder means reversing the disorder's progression, ideally to the point of eliminating the disorder itself.

### EMBODIMENTS OF THE INVENTION

RAGE signaling is a key process in cell activation (e.g., in diabetic vasculature). Experiments, whose data are set forth herein, have demonstrated that RAGE tail (i.e., the cytoplasmic domain of RAGE) interacts with Diaphanous, a key molecule involved in signaling and motility. The experiments include immunoprecipitation and confocal microscopy.

Specifically, this invention provides a polypeptide consisting essentially of all or a portion of the cytoplasmic domain of RAGE. In the preferred embodiment, the RAGE is human RAGE. In another embodiment, the polypeptide is isolated. In one embodiment, the portion of the cytoplasmic domain of RAGE is at least 4 amino acid residues in length, and preferably more than 7 amino acid residues in length. In another embodiment, the portion consists essentially of one of the following fragments of the 41 amino acid residue human cytoplasmic domain of RAGE (wherein for this example only, the residue numbering is 1 through 41, with the number 1 representing the amino end of the cytoplasmic domain): (a) 1-5; (b) 6-10; (c) 11-15; (d) 16-20; (e) 21-25; (f) 26-30; (g) 31-35; (h) 36-41; (i) 1-10); (j) 11-20; (k) 21-30; (l) 31-41; (m) 1-14; (n) 15-28; (o) 29-41; (p) 1-21; and (q) 22-41.

This invention further provides a pharmaceutical composition comprising (a) all or a portion of the cytoplasmic domain of RAGE and (b) a pharmaceutically acceptable carrier.

This invention further provides a polypeptide consisting essentially of a portion of Diaphanous that binds to the cytoplasmic domain of RAGE. In one embodiment, the polypeptide consists essentially of all or a portion of the FH1 domain of Diaphanous. The FH1 domain of Diaphanous corresponds to residues 570-735 of the human Diaphanous amino acid sequence. In one embodiment, the portion of the FH1 domain of Diaphanous is at least 4 amino acid resides long, and preferably more than 7 amino acid residues in length. Examples of a portion of the FH1 domain of Diaphanous include, but are not limited to, amino acid residues 570-610, amino acid residues 611-660, amino acid residues 661-700 and amino acid residues 701-735. In the preferred embodiment, the Diaphanous is human Diaphanous. In another embodiment, the polypeptide is isolated.

This invention further provides a pharmaceutical composition comprising (a) a portion of Diaphanous that binds to the cytoplasmic domain of RAGE and (b) a pharmaceutically acceptable carrier.

This invention further provides a nucleic acid that encodes a polypeptide consisting essentially of all or a portion of the cytoplasmic portion of RAGE. In the preferred embodiment, the RAGE is human RAGE. In another embodiment, the nucleic acid is isolated.

This invention further provides a nucleic acid encoding a polypeptide consisting essentially of a domain of Diaphanous that binds to the cytoplasmic domain of RAGE. In one embodiment, the polypeptide consists essentially of all or a portion of the FH1 domain of Diaphanous. In the preferred embodiment, the Diaphanous is human Diaphanous. In another embodiment, the nucleic acid is isolated.

This invention further provides an expression vector comprising a nucleic acid that encodes a polypeptide consisting essentially of all or a portion of the cytoplasmic domain of RAGE. This invention further provides a cell comprising the expression vector. In one embodiment, the cell is a bacterial, amphibian, yeast, fungal, insect, or mammalian cell.

This invention further provides an expression vector comprising a nucleic acid that encodes a polypeptide consisting essentially of a domain of Diaphanous that binds to the cytoplasmic domain of RAGE. This invention further provides a cell comprising the expression vector. In one embodiment, the cell is a bacterial, amphibian, yeast, fungal, insect, or mammalian cell.

This invention further provides a method for inhibiting binding between Diaphanous and the cytoplasmic domain of RAGE comprising contacting Diaphanous and the cytoplasmic domain of RAGE with an agent that, under suitable conditions, inhibits binding therebetween.

In one embodiment, the agent is a polypeptide consisting essentially of all or a portion of the cytoplasmic domain of RAGE. In the preferred embodiment, the RAGE is human RAGE. In another embodiment, the polypeptide is isolated.

In another embodiment, the agent is a polypeptide consisting essentially of a portion of Diaphanous that binds to the cytoplasmic domain of RAGE. In another embodiment, the polypeptide consists essentially of all or a portion of the FH1 domain of Diaphanous. In the preferred embodiment, the Diaphanous is human Diaphanous. In another embodiment, the polypeptide is isolated.

In another embodiment, the agent is a mimetic of (i) a polypeptide consisting essentially of all or a portion of the cytoplasmic domain of RAGE or (ii) a polypeptide consisting essentially of a portion of Diaphanous that binds to the cytoplasmic domain of RAGE. A mimetic can be, but is not limited to, a small molecule mimic of the polypeptide consisting essentially of all or a portion of the cytoplasmic domain of RAGE, or a small molecule mimic of the polypeptide consisting essentially of a portion of Diaphanous that binds to the cytoplasmic domain of RAGE. The mimetic may have increased stability, efficacy, potency and bioavailability. Furthermore, the mimetic may also have decreased toxicity, and/or enhanced mucosal intestinal permeability. The mimetic may be synthetically prepared.

This invention further provides a method for identifying an agent that inhibits binding between Diaphanous and the cytoplasmic domain of RAGE comprising (a) contacting Diaphanous and the cytoplasmic domain of RAGE with the agent under conditions that would permit binding between Diaphanous and the cytoplasmic domain of RAGE in the absence of the agent, (b) after a suitable period of time, determining the amount of Diaphanous bound to the cytoplasmic domain of RAGE and (c) comparing the amount of Diaphanous bound to the cytoplasmic domain of RAGE determined in step (b) with the amount of Diaphanous bound to the cytoplasmic domain of RAGE in the absence of the agent, whereby a lower amount of binding in the presence of the agent indicates that the agent inhibits the binding between Diaphanous and the cytoplasmic domain of RAGE.

In one embodiment, the agent is selected from the group consisting of a polypeptide, a nucleic acid and an organic molecule.

One example of a method for identifying an agent that inhibits binding between Diaphanous and the cytoplasmic domain of RAGE is set forth below.

Epitope-tagged full length Diaphanous and then domains of Diaphanous, such as the FH1 domain, can be tagged with, for example, his tags. At the same time, GST-labeled RAGE cytosolic domain and then subcomponents of the cytosolic domain can be generated. These materials can be generated in bacteria, for example. His tags bind to Nickel columns and his-tagged Diaphanous and domains of Diaphanous can be expressed and bound to the nickel column. Bacterial lysates expressing GST RAGE cytosolic domain or subdomains can be chromatographed onto the Nickel columns containing the his-tagged Diaphanous constructs. After washing to remove nonspecific binding, the his-tagged epitopes and their bound materials can be released from the nickel column, and gels/western blots using antibodies to GST can be used to identify binding of RAGE cytosolic domain to his-Diaphanous. Negative controls can include empty his and empty GST tags.

Finally, this invention provides a method for treating a RAGE-related disorder in a subject afflicted therewith comprising administering to the subject a therapeutically effective amount of an agent that inhibits the binding between Diaphanous and the cytoplasmic domain of RAGE. In one embodiment, the disorder is selected from the group consisting of atherosclerosis, multiple sclerosis, systemic lupus erythematosus, sepsis, transplant rejection, asthma, arthritis, tumor growth, cancer, metastases, complications due to diabetes, retinopathy, neuropathy, nephropathy, impotence, impaired wound healing, gastroparesis, Alzheimer's disease, Huntington's disease, amyotrophic lateral sclerosis, neointimal formation, amyloid angiopathy, inflammation, glomerular injury, and seizure-induced neuronal damage. In the preferred embodiment, the subject is human.

In another embodiment, the agent is a polypeptide consisting essentially of all or a portion of the cytoplasmic domain of RAGE. In the preferred embodiment, the RAGE is human RAGE. In another embodiment, the polypeptide is isolated.

In another embodiment, the agent is a polypeptide consisting essentially of a portion of Diaphanous that binds to the cytoplasmic domain of RAGE. In one embodiment, the polypeptide consists essentially of all or a portion of the FH1 domain of Diaphanous. In the preferred embodiment, the Diaphanous is human Diaphanous.

### REFERENCES

1. Castrillon, D. H., and Wasserman, S. A., “Diaphanous is required for cytokinesis in Drosophila and shares domains of similarity with the products of the limb deformity gene,” Development 120:3367-3377, 1994.
2. Lynch, E. D., Lee, M. K., Morrow, J. E., Welcsh, P. L., Leon, P. E., and King, M. C., “Nonsyndromic deafness DFNA1 associated with mutation of a human homolog of the Drosophila gene Diaphanous,” Science 278:1315-1318, 1997.
3. Wasserman, S., “FH proteins as cytoskeletal organizers,” Trends Cellular Biology 8:111-115, 1998.
4. Watanabe, N., Madaule, P., Reid, T., Ishizaki, T., Watanabe, G., Kakizuka, A., Saito, Y., Nakao, K., Jockusch, B. M., and Narumiya, S., “P140mDia, a mammalian homolog of Drosophila Diaphanous, is a target protein for Rho small GTPase and is a ligand for profiling,” EMBO Journal 16:3044-3056, 1997.
5. Narumiya, S., Ishizaki, T., and Watanabe, N., “Rho effectors and reorganization of the actin cytoskeleton,” FEBS Letters 410:68-72, 1997.
6. Nakano, K., Takaishi, K., Kodama, A., Mammoto, A., Shiozaki, H., Monden, M., and Takai, Y., “Distinct actions and cooperative roles of ROCK and mDia in Rho small G Protein-induced reorganization of the actin cytoskeleton in Madin-Darby Canine Kidney Cells,” Molecular Biology of the Cell 10:2481-2491, 1999.
7. Westendorf, J. J., Mernaugh, R., and Hiebert, S. W., “Identification and characterization of a protein containing formin homology (FH1/FH2) domains,” Gene 232: 173-182, 1999.
8. Kato, T., Watanabe, N., Morishima, Y., Fujita, A., Ishizaki, T., and Narumiya, S., “Localization of a mammalian homolog of Diaphanous, mDia1, to the mitotic spindle in HeLa cells,” Journal of Cell Science 114:775-784, 2000.
9. Afshar, K., Stuart, B., and Wasserman, S. A., “Functional analysis of the Drosophila Diaphanous FH protein in early embryonic development,” Development 127:1887-1897, 2000.
10. Watanabe, N., Kato, T., Fujita, A., Ishizaki, T., and Narumiya, S., “Cooperation between mDia1 and ROCK in Rho-induced actin reorganization,” Nature Cell Biology 1:136-143, 1999.
11. Krebs, A., Rothkegel, M., Klar, M., and Jockusch, B. M., “Characterization of functional domains of mDia1, a link between the small GTPase Rho and the actin cytoskeleton,” Journal of Cell Science 114:3663-3672, 2001.
12. Riveline, D., Zamir, E., Balaban, N. Q., Schwarz, U.S., Ishizaki, T., Narumiya, S., Kam, Z., Geiger, B., and Bershadsky, A. D., “Focal contacts as mechanosensors: externally applied local mechanical force induces growth of focal contacts by an mDia-1 dependent and ROCK-independent mechanism,” Journal of Cell Biology 153:1175-1185, 2001.
13. Ishizaki, T., Morishima, Y., Okamoto, M., Furuyashiki, T., Kato, T., and Narumiya, S., “Coordination of microtubules and the actin cytoskeleton by the Rho effector mDia1,” Nature Cell Biology 3:8-14, 2001.
14. Tsuji, T., Ishizaki, T., Okamoto, M., Higashida, C., Kimura, K., Furuyashiki, T., Arakawa, Y., Birge, R. B., Nakamoto, T., Hirai, H., and Narumiya, S., “ROCK and mDia1 antagonize in Rho-dependent Rac activation in Swiss 3T3 fibroblasts,” Journal of Cell Biology 157:819-830, 2002.
15. Sahai, E., and Marshall, C. J., “ROCK and dia have opposing effects on adherens junctions downstream of Rho,” Nature Cell Biology 4:408-415, 2002.
16. Geneste, O., Copeland, J. W., and Treisman, R., “LIM kinase and Diaphanous cooperate to regulate serum response factor and actin dynamics,” Journal of Cell Biology 157:831-838, 2002.
17. Ganguly, A., and Lohia, A., “The Diaphanous protein from Entamoeba histolytica controls cell motility and cytokinesis,” Archives of Medical Research 31:S137-S139, 2000.
18. Watanabe, N., Madaule, P., Reid, T., Ishizaki, T., Watanabe, G., Kakizuka, A., Saito, Y., Nakao, K., Jockusch, B. M., and Narumiya, S., “P140mDia, a mammalian homolog of Drosophila Diaphanous, is a target protein for Rho small GTPase and is a ligand for profiling,” EMBO Journal 16:3044-3056, 1997.
19. Palazzo, A. F., Eng, C. H., Schlaepfer, D. D., Marcantonio, E. E., and Gundersen, G. G., “Localized stabilization of microtubules by integrin- and FAK-facilitated Rho signaling,” Science 303:836-839, 2004.
20. Li, F., Higgs, H. N., “Dissecting requirements for autoinhibition of actin nuceartion by the formin, mDia1,” J Biol Chem 280:6986-6992, 2005.
21. Vicente-Manzanares, M., Rey, M., Perez-Martinez, M., Yanez-Mo, M., Sancho, D., Cabrero, J. R., Barriero, O., de la Fuente, H., Itoh, K., Sanchez-Madrid, F., “The rho A effector mDia is induced during T cell activation and regulates actin polymerization and cell migration in t lymphocytes,” J Immunology 171:1023-1034, 2003.
22. Arakawa, Y. Bito, H., Furuyashiki, T., Tsuji, T., Takemoto-Kimura, S., Kimura, K., Nozaki, K., Hashimoto, N., and Narumiya, S., “Control of axon elongation via an SDF-1 alpha/rho/mdia pathway in cultured cerebellar granule neurons,” J Cell Biology 161:381-391, 2003.
23. Bucciarelli, et al., Circulation 106:2827-2835, 2002.
24. Wendt, T. M., et al., Am. J. Pathol. 162:1123-1137, 2003.
25. Cipollone, F., Circulation 108:1070-1077, 2003.
26. Taguchi, A., Nature 405:354-360, 2000.
27. Sakaguchi, et al., J. Clin. Invest. 111:959-972, 2003.

1. An isolated polypeptide consisting essentially of all or a portion of the cytoplasmic domain of a RAGE protein.

2. The polypeptide of claim 1, wherein the RAGE protein is human RAGE.

3. (canceled)

4. A pharmaceutical composition comprising the polypeptide of claim 1 and a pharmaceutically acceptable carrier.

5. A polypeptide consisting essentially of all or a portion of the FH1 domain of a Diaphanous protein.

6. (canceled)

7. The polypeptide of claim 5, wherein the Diaphanous protein is human Diaphanous.

8. (canceled)

9. A pharmaceutical composition comprising the polypeptide of claim 5 and a pharmaceutically acceptable carrier.

10. An isolated nucleic acid that encodes a polypeptide consisting essentially of all or a portion of the cytoplasmic domain of a human RAGE protein.

11.-13. (canceled)

14. An isolated nucleic acid encoding a polypeptide which consists essentially of all or a portion of the FH1 domain of a human Diaphanous protein.

15. (canceled)

16. (canceled)

17. An expression vector comprising the nucleic acid of claim 10.

18. A cell comprising the expression vector of claim 17.

19. The cell of claim 18, wherein the cell is a bacterial, amphibian, yeast, fungal, insect, or mammalian cell.

20. An expression vector comprising the nucleic acid of claim 14.

21. A cell comprising the expression vector of claim 20.

22. The cell of claim 21, wherein the cell is a bacterial, amphibian, yeast, fungal, insect, or mammalian cell.

23. A method for inhibiting binding between a human Diaphanous protein and a human RAGE protein comprising contacting the Diaphanous protein and the RAGE protein with a polypeptide consisting of (a) all or a portion of the cytoplasmic domain of the human RAGE protein or (b) all or a portion of the FH1 domain of the human Diaphanous protein.

24-39. (canceled)
